(19) United States
(12) Patent Application Publication
(10) Pub. No.: US 2006/0153938 A1
(43) Pub. Date: Jul. 13, 2006

(54) Eye -Rinse Solution Made of Grape juice

(76) Inventor: Irwin Elias Komery, (US)

Correspondence Address: Irwin E. Komery, 509 Rue Pasteur, Sherbrooke, QC, J1J 2T5, CA

(21) Appl. No.: 10/543,164

(22) PCT Filed: Oct. 20, 2003
(86) PCT No.: PCT/CA03/01587

§ 371 Date: Jul. 21, 2005

(30) Foreign Application Priority Data
Jun. 20, 2003 (CA) 2431683

(51) Int. Cl. A61K 36/87

(52) U.S. Cl. 424/766

(57) ABSTRACT

This is an eye-rinse remedy solution which relieves migraine headaches. There are two ingredients to this solution. They are unpolluted, filtered or distilled water and the juice from the fresh grapes. The preferable solution is one part water (H₂O) to the one-half part to one part juice from fresh grapes. The method of use is to allow and apply two drops of the solution onto both eyes and blinking several times. Then wipe away the excess. Application of the solution onto the eyes is usually done once but may be applied as many times again if the migraine headache, headache or other five claims persist.

### INTRODUCTION

This is an eye-rinse remedy solution which relieves the person from migraine headache and headache and five other claims as submitted, in the six claims.

It is a very simple solution, but its effects and beneficiary remedies are marvellous.

There are no known secondary, ill or harmful side effects of the solution to the eyes, brain or body, when applied to the eyes.

This eye-rinse remedy solution has been tried since 1987 without any ill or harmful side effects.

### SOLUTION

There are two natural ingredients in this solution. They are unpolluted, filtered or distilled water and the juice form fresh grapes. The amounts of water and juice may vary. The preferable solution is one part water to one-half part to one part juice form fresh grapes.

### METHOD OF USE

Allowing and applying two drops of the solution onto both eyes and blinking the eyes. Then wipe away the excess solution. The application of the solution is usually done once, but may be reapplied as many times as necessary if the migraine headache or five other claims persist.

### PROPERTIES AND USES OF THE EVE-RINSE REMEDY SOLUTION

1. To clear and clean the eyes;
2. The relieve the eyes from tiredness and stress;
3. To relieve the person from mental stress and from slight depression;
4. To relieve the pain of the eyes from headaches and migraine headaches;
5. To relieve the person from migraine headaches and simple headaches;
6. To relieve the person from pain at the back of the neck and of the muscles at the back of the neck due to stress, tiredness and/or from arthritis of the cervical spine.

### SHELF LIFE

As the shelf life of this eye-rinse remedy solution has not been favourable, it is to be noted that mold appears after 24 hours exposure to room temperature. It takes approximately one month for mold to appear when refrigerated. Probable possibilities to prolong shelf life are as follow:—
1. Refrigeration;
2. Freeze drying the juice from grapes into powder and which then can be mixed with water;
3. Pasteurising;
4. Preferably adding ingredient preservative to prevent alteration or molding if left at room temperature for indefinite time and which does not affect the eyes. This is the preferable solution.

### CONCLUSION

This eye rinse remedy solution is a revolutionary method with astounding results for the relief of migraine headaches and simple headaches and five other claims as submitted.

1. A composition of one part unpolluted water to one-half part to one part juice to clear and clean the eyes.

2. A composition of one part unpolluted water to one-half to one part juice for removing tiredness and/or stress of the eyes.

3. A composition of one part unpolluted water to one-half part to one part juice for relieving the person from mental stress and sight depression.

4. A composition of one part unpolluted water to one-half to one part juice for relieving pain in the eyes caused from the headaches and/or migraine headaches.

5. A composition of one part unpolluted water to one-half part to one part juice for the very important property of relieving the person from migraine headaches and/or simple headaches.

6. A composition of one part unpolluted water to one-half part to one part juice for relieving the person from pain at the back of the neck and/or of the muscles at the back of the neck due to stress or tiredness and/or arthritis of the cervical spine.
